(19) United States
(12) Patent Application Publication
(10) Pub. No.: US 2004/0171911 A1
(43) Pub. Date: Sep. 2, 2004

(54) Pneumatic penile prosthesis

(76) Inventor: Victor M. Zurita, Rolling Meadows, IL (US)

Correspondence Address: LAWRENCE CRUZ, 12 GOOD HILL RD, BETHEL, CT, 06801, US

(21) Appl. No.: 10/374,335

(22) Filed: Feb. 27, 2003

(51) Int. Cl. A61F 5/00

(52) U.S. Cl. 600/38

(57) ABSTRACT

A penile prosthesis is adapted to be worn over the patient's penis, has the visual appearance of an erect penis and is comprised of two parts that are of generally the same length. The two parts are generally tubular shaped and are preferably made of a semi-rigid plastic material. The two parts are adapted to be selectively joined together and disassembled, preferably by a threaded connection. A valve permits selective de-pressurization of an annular space formed around the penis inside the prosthesis, thereby applying vacuum pressure to maintain the prosthesis in position.

### RELATED APPLICATIONS

None.

### TECHNICAL FIELD

The present invention relates to devices for treating erectile dysfunction and, more particularly, to a prosthesis device worn over the penis for treating erectile dysfunction.

### BACKGROUND OF THE INVENTION

A large number of males experience various types and varying degrees of erectile dysfunction. The level of dysfunction ranges from complete and irreversible loss of function of the type experienced by paraplegics or individuals who have had partial loss of penile tissue due to surgery or trauma, to partial loss of function including premature ejaculation or incomplete erection.

Various known devices exist for treating partial loss of function, including vacuum devices. Other known devices include surgically implanted prostheses or structural components. Such known devices are often inconvenient, uncomfortable, too noticeable in the visual or tactile sense, and relatively ineffective. Surgical implants, in particular, are often expensive and subject the patient to complications.

### OBJECTS OF THE INVENTION

It is an object of the present invention to provide a device and associated method of use for treating erectile dysfunction while overcoming the shortcomings of known devices mentioned above and other existing devices.

It is a further object to provide a relatively inexpensive, uncomplicated, yet effective device and associated method for treating erectile dysfunction.

These objects as well as others are achieved by the present invention described below.

### SUMMARY OF THE PRESENT INVENTION

The present invention is directed to a penile prosthesis adapted to successfully treat most types and degrees of erectile dysfunction. The prosthesis, adapted to be worn over the patient's penis, has the visual appearance of an erect penis. In the preferred embodiment, it is comprised of two parts that are of generally the same length. The two parts are generally tubular shaped and are preferably made of a semi-rigid plastic material. The two parts are adapted to be selectively joined together and disassembled, preferably by a threaded connection through relative rotation.

A first one of the parts is open at both ends and is positioned over the penis so that its proximal end is in abutment with the pubis. The first part has an interior ridge on the inner circumferential wall, thereby forming a concentric ring of slightly smaller diameter than the inner circumferential wall. The ring serves as a sealing surface in contact with the skin of the penis

The second part is open at one end, and has a generally closed opposite end that has a small one-way valve. The closed end is rounded and anatomically resembles the glans penis. The open end, which has a threaded interface mates with threads on the distal end of the first part to enclose the patient's penis.

Once the first and second parts are positioned over the penis, suction or vacuum pressure is applied through the valve, securing the assembled device in place over the penis.

### BRIEF DESCRIPTION OF THE DRAWINGS

FIG. 1 is a perspective view of a preferred embodiment of the present invention penile prosthesis.

FIG. 2 is side, cross-sectional view of the preferred embodiment of the invention.

FIG. 3 is a partial, perspective view of a first component of the preferred embodiment of the present invention.

FIG. 4 is a perspective view of a second component of the preferred embodiment of the present invention.

### DETAILED DESCRIPTION OF THE PREFERRED EMBODIMENT

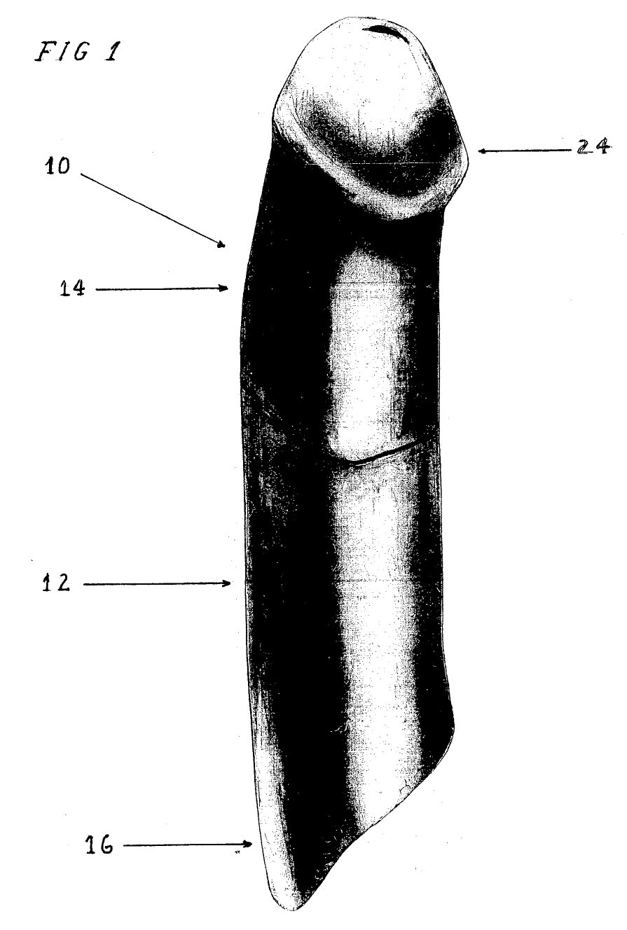

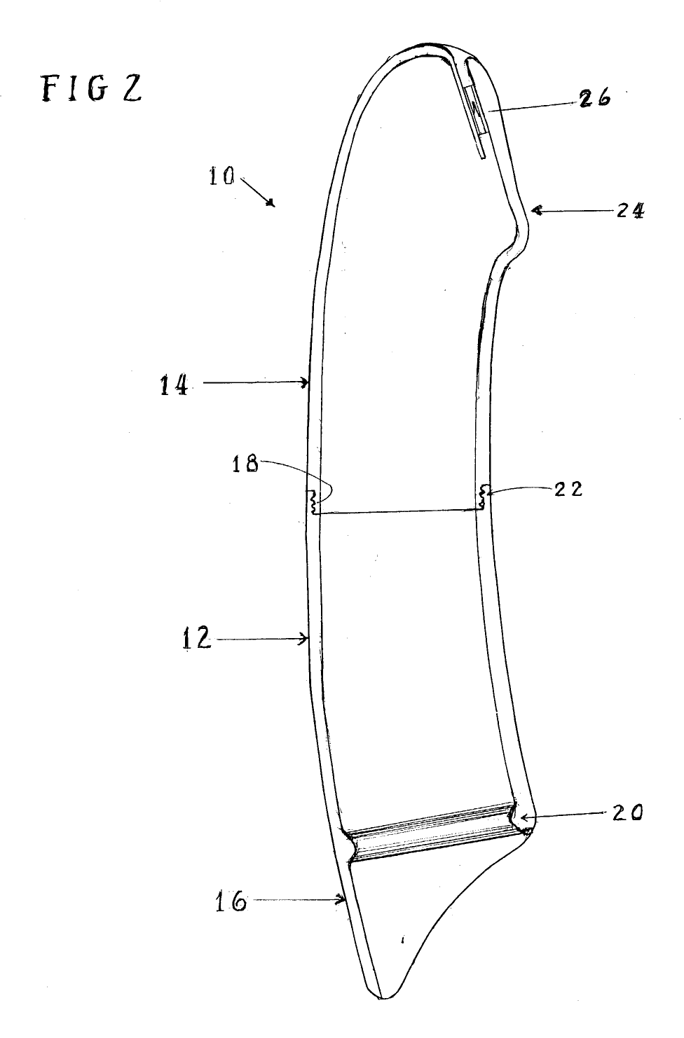

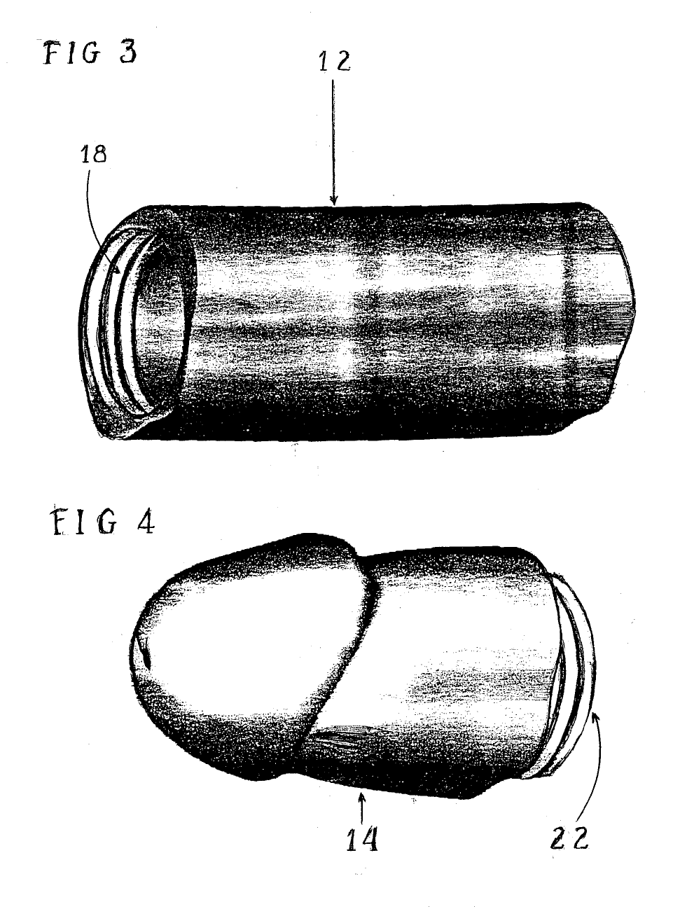

A prosthesis (10) according to a preferred embodiment of the present invention is shown in FIGS. 1-4. The prosthesis comprises a first section (12) and a second section (14). The first section (12), which is generally tube-shaped and thus has a central passage therethrough, has at its proximal end an elongated inferior portion (16). At its distal end, the first section (12) has a threaded section (18). The first section further includes an interior ridge (20) forming an internal, concentric ring.

The prosthesis second section (14) is generally tube-shaped and thus has a central passage therethrough. At its proximal end, the second section (14) has a threaded section (22). The distal end of the second section (14) is closed and has exterior contours (24) adapted to resemble the natural exterior of a penis. The second section (14) further includes a one-way valve (26) at the tip. The valve (26) may be any one of various type valves generally known.

In use, the first section (12) is positioned around a patient's penis. The elongated inferior portion (16) is placed in contact with the scrotum and the perineum to serve as a support point and to maintain position of the first section (12) relative to the patient's body. The interior ridge (20) is sized to surround the penis snugly, but not uncomfortably tightly. The penis is then pulled through the first section (12) to ensure that the first section (12) is in position against the scrotum and the perineum and that the penis extends distally past the first section (12). The second section (14) is brought down over the penis so that the threaded section (22) engages and mates with the threaded section (18) of the first section (12). Once the first and second sections (12, 14) are fastened over the penis, suction or vacuum pressure from an external source (not shown) is applied through the valve (26) to the interior of the prosthesis (10). The annular seal formed by the contact line between the interior ridge (20) and the outside of the penis contains the vacuum pressure distally of the ridge (20) in the annular space around the outside of the penis. The vacuum pressure serves to maintain the prosthesis in position in a comfortable and well-supported manner during sexual activity.

Removal of the prosthesis (10) may be effected by disassembling the first and second sections. Under preferred conditions, the vacuum pressure will not be great enough to cause discomfort or difficulty in disassembling the first and second sections. Alternatively, if it is desired or necessary, the valve (26) can be engaged to release the vacuum pressure prior to disassembly.

The two sections of the prosthesis (10) may be made of any suitable semi-rigid or rigid plastic, elastomer or composite. The exterior and interior surfaces may, if desired, be smooth or have various surface textures including ridges, bumps or other variations to enhance comfort, sensation, fit, durability or other characteristics. Various coatings may be used on the exterior or the interior, such as rubberized or lubricating coatings. The prosthesis (10) may also be made of laminate or layered construction incorporating layers of rubber, foam, inflatable chambers, varying textures or other desirable features.

What is claimed is:

1) A penile prosthesis comprising
a generally tubular shaped body having an open proximal end and a closed distal end, and being adapted to fit over a penis;
a sealing surface on the interior of said body being adapted to engage the skin of said penis in a sealing manner;
an annular chamber inside said body and around said penis, between said sealing surface and said distal end: and
a valve in fluid communication with said annular chamber and adapted to selectively facilitate change in pressure in said annular chamber relative to ambient pressure.

2) A prosthesis according to claim 1, wherein
said body comprises a first portion and a second portion, wherein said first portion and said second portion are adapted to be selectively assembled and disassembled.

3) A prosthesis according to claim 2, wherein
said first portion comprises a generally tube-shaped body having an open proximal end and an open distal end; and
said second portion comprises an open proximal end and a closed distal end, said first and second portions being adapted to be assembled and disassembled at said open distal end of said first portion and said open proximal end of said second portion.

4) A prosthesis according to claim 3, wherein
said sealing surface is located on said first portion and said valve is located on said second section.

5) A prosthesis to claim 3, further comprising
a first threaded section located at said open distal end of said first portion and a second threaded section located at said open proximal portion of said second portion, said first and second threaded sections being selectively engageable and disengageable to assemble and disassemble said first and second portions.

6) A prosthesis to claim 1, further comprising
an elongated inferior section located at said proximal end and adapted to contact the scrotum and perineum when said prosthesis is worn over the penis.

7) A method of using a penile prosthesis, said method comprising
positioning over the penis a generally tubular shaped body having an open proximal end and a closed distal end, a sealing surface on the interior of said body being adapted to engage the skin of said penis in a sealing manner, an annular chamber inside said body and around said penis, between said sealing surface and said distal end, and a valve in fluid communication with said annular chamber; and
activating said valve to facilitate a change in pressure in said annular chamber relative to ambient pressure.

8) A method prosthesis to claim 7, wherein
said change in pressure is a negative change, resulting in vacuum pressure within said annular chamber.

9) A method prosthesis to claim 7, wherein
said tubular shaped body comprises a first portion comprising a generally tube-shaped body having an open proximal end and an open distal end, and a second portion comprising an open proximal end and a closed distal end; and
said method further comprises selectively assembling said open distal end of said first portion and said open proximal end of said second portion together after positioning said first portion over said penis.

10) A method prosthesis to claim 9, wherein
said first and second portions are assembled by rotating one relative to the other, thereby causing respective threaded sections on each to engage and matingly fit.
